(19) United States
(12) Patent Application Publication
(10) Pub. No.: US 2008/0069788 A1
(43) Pub. Date: Mar. 20, 2008

(54) Basic Aluminium Halogenide Complexes

(76) Inventor: Norbert Roesch, Gersthofen (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/594,176

(22) PCT Filed: Mar. 22, 2005
(86) PCT No.: PCT/EP05/03003

§ 371 Date: Aug. 13, 2007

(30) Foreign Application Priority Data
Mar. 26, 2004 (DE) 10 2004 015 025.7

(51) Int. Cl. A61K 8/26; A61Q 15/00; C07F 5/06

(52) U.S. Cl. 424/68; 556/186

(57) ABSTRACT

Basic aluminum halide complexes obtained by reaction of aluminum metal with hydrohalic acid and an organic acid, where either the aluminum metal is dissolved in a mixture of hydrohalic acid and organic acid, or the aluminum metal is firstly dissolved in the hydrohalic acid and then the organic acid is added.

Basic aluminum halides in the form of chlorides are used as active ingredient in cosmetic preparations such as antiperspirants or astringents. The much more effective aluminum chloride hexahydrate is ruled out for this application due to its skin-irritative effect. It is used for therapeutic purposes in cases of hyperhidrosis (excessive sweating). The difference between the specified aluminum chlorides is that the basic salts form oligomeric and polymeric aluminum species (cf. S. Schönherr, H. Görz, D. Müller and W. Gessner; in Z. anorg. alig. Chem. 476, 188-194 (1991)), which display reduced effectiveness in antiperspirants.

For many years attempts have therefore been made to increase the effectiveness of the basic aluminum chlorides while retaining the good skin compatibility. The patents EP 0 308 937; EP 0 183 171; U.S. Pat. No. 4,359,456; EP 0 191 628 describe basic aluminum halides, preferably chlorides, which are subjected to heat treatment. The resulting solutions were analyzed using gel permeation chromatography (GPC) with regard to their polymer distribution. The heat-treated samples exhibit degradation of polymeric species and thus an increased effectiveness in antiperspirants. However, it is also known from the literature that the distributions of the various polymeric species are reversible equilibria, and therefore the activation is lost over time in protic solvents.

Another way which has been described for synthesizing more effective antiperspirant active ingredients is the use of mixed compounds which consist of combinations of basic aluminum halides (preferably chlorides), zirconium salts and glycine. Various preparation methods are given in U.S. Pat. No. 4,871,525; U.S. Pat. No. 4,775,528 and DE 25 37 359.

A further increase in the effectiveness of the zirconium-aluminum mixed salts is described in EP 0 653 203; WO 02/34223; WO 0234223; EP 0 455 489 and EP 0 444 564. However, a disadvantage of the zirconium-aluminum salts is the high cost of the obtainable zirconium raw materials.

EP 1 364 651 describes the use of a mixture of basic aluminum hydrate and trivalent aluminum salts which contain organic ligands for producing pharmaceutical preparations which have a skin-deswelling effect. A use as antiperspirant active ingredient has not already been described therein, nor can the person skilled in the art derive such an antiperspirant effect from this specification. In the CTFA database, the Na salt of a complex of lactic acid and aluminum chlorohydrate (Choracel) and its effect as cosmetic stringent is known. However, this product is not a true aluminum-lactic acid complex, but a mixture of aluminum chlorohydrate and lactic acid. Other complexes of aluminum salts, possibly zirconium salts, and hydroxy acids are described in U.S. Pat. No. 3,542,919, U.S. Pat. No. 3,553,316 and U.S. Pat. No. 3,991,176. However, in the preparation of these products, the hydroxyl acid is added to the finished aluminum salt, and not, as is the case in the present invention, during the preparation of the aluminum salt. Consequently, the addition of the hydroxy acid in this prior art can have no influence on the distribution of the polymeric fractions.

The object of the present invention is to improve the effectiveness of basic aluminum chlorides as antiperspirant active ingredient through reduced formation of polymeric aluminum structures. This object was achieved by the incorporation of organic acids as complex ligands into the basic aluminum halides.

The invention provides basic aluminum halide complexes obtained by reaction of aluminum metal with hydrohalic acid and an organic acid, where either the aluminum metal is dissolved in a mixture of hydrohalic acid and organic acid, or the aluminum metal is firstly dissolved in the hydrohalic acid and then the organic acid is added.

Here, the first variant, i.e. the dissolution of the aluminum metal in the mixture of hydrohalic acid and organic acid, is preferred.

Suitable hydrohalic acid is HBr, but in particular HCl, in the form of its aqueous solution. The concentration of the hydrohalic acid is preferably 1 to 10% by weight, preferably 5% by weight.

Suitable organic acids are in particular those which contain 1 to 5 carbon atoms. These organic acids can be mono-, di- or tricarboxylic acids, but aminocarboxylic acids or hydroxycarboxylic acids are also suitable, with the latter being preferred. Examples of such acids are glycolic acid, lactic acid, tartronic acid, tartaric acid, malic acid and glycine.

The ratio of hydrohalic acid to organic acid can be between 10 and 90 mol % for each of the two acids. Preference is given to a mixture of 20 to 50 mol % of hydrohalic acid and 50 to 80 mol % of organic acid. The concentration of the hydrohalic acid here is as stated above.

The aluminum metal is then dissolved in this acid mixture. The aluminum metal here can be in the form of powder, grit, granules or bars. The reaction preferably takes place at temperatures of from 20 to 100° C., preferably at 50 to 100° C. The reaction proceeds until the desired atomic ratio of Al to halogen or to the anion of the organic acid is established in the reaction solution.

The resulting aqueous solution of the basic aluminum halide can be used directly as antiperspirant active ingredient. In order to set certain ligand ratios in the complex, it is, however, possible to also additionally add hydrohalic acid and/or organic acid after the reaction between aluminum metal and the acid mixture.

In addition, it is advantageous to adjust the aqueous solution of the basic aluminum complex to a pH of from about 4 to 5 by adding a buffer substance. Suitable buffer substances are, in particular, α-amino acids, for example glycine.

The resulting aqueous solutions of the basic aluminum complexes are characterized in that the fractions of Al polymer are significantly reduced, while at the same time the fraction of the Al monomers and Al oligomers increases. These solutions can be further processed directly as antiperspirant active ingredient. However, it is also possible to mix these basic aluminum complexes according to the invention with other customary antiperspirant active ingredients, for example with Al-zirconium-glycine chlorocomplexes or other metal complexes which contain Zr, Hf, Ti or Sn. In the case of this variant too, it may be advantageous to add a buffer substance, for example an amino acid such as glycine, in order to set a pH of 4-5.

### EXAMPLES

### Example 1

In a column-like vessel of length 60 cm and diameter 9 cm with jacket heating and attached condenser, about 2.5 kg of pieces of aluminum in the form of small bars having in each case a weight of about 11 g were initially introduced and pickled with dilute hydrochloric acid. A 1:1 mixture of hydrochloric acid and glycolic acid was then poured in. After charging the solution and heating to about 100° C., the aluminum metal was attacked with the evolution of hydrogen. The progress of the reaction was observed by determining the aluminum and chloride content of the solution. At an aluminum:chlorine atomic ratio of 3.3:1, the reaction was terminated by discharging the solution. The resulting aluminum complex was used for active ingredients described in Examples 3 and 6.

### Example 2

In a column-like vessel of length 60 cm and diameter 9 cm with jacket heating and attached condenser, about 2.5 kg of pieces of aluminum in the form of small bars having in each case a weight of about 11 g were initially introduced and pickled with dilute hydrochloric acid. A 1:1 mixture of hydrochloric acid and lactic acid was then poured in. After charging the solution and heating to about 100° C., the aluminum metal was attacked with the evolution of hydrogen. The progress of the reaction was observed by determining the aluminum and chloride content of the solution. At an aluminum:chlorine atomic ratio of 3.3:1, the reaction was terminated by discharging the solution. The resulting aluminum complex was used for active ingredients described in Examples 4, 5 and 7.

### Example 3

The aluminum complex prepared in Example 1 was mixed with hydrochloric acid, water and glycine in order to obtain the composition given in Table 1.

TABLE 1

| Fraction | Active ingredient 1 |
|---|---|
| Aluminum % (w/w) | 8.00 |
| Chlorine % (w/w) | 3.27 |
| Glycolate % (w/w) | 6.94 |
| Glycine | 7.02 |

### Example 4

The aluminum complex prepared in Example 2 was mixed with hydrochloric acid and water in order to obtain the composition given in Table 2.

TABLE 2

| Fraction | Active ingredient 2 |
|---|---|
| Aluminum % (w/w) | 8.00 |
| Chlorine % (w/w) | 3.33 |
| Lactate % (w/w) | 8.37 |
| Glycine | — |

### Example 5

The aluminum complex prepared in Example 2 was mixed with hydrochloric acid, water and lactate in order to obtain the composition given in Table 3.

TABLE 3

| Fraction | Active ingredient 3 |
|---|---|
| Aluminum % (w/w) | 8.00 |
| Chlorine % (w/w) | 3.27 |
| Lactate % (w/w) | 8.21 |
| Glycine | 7.00 |

The three complexes described in the examples were analyzed with regard to their molecular weight distribution using GPC measurements. The separating column used was an RP1 system. The eluent used was HNO₃/water, the change in the refractive index being detected.

The reference substance is standard commercial basic aluminum chloride and aluminum chloride hexahydrate.

Basic aluminum chloride shows four marked peaks at a maximum at:

TABLE 4

| Peak | Retention time |
|---|---|
| 1 | 5.3 min |
| 2 | 6.1 min |
| 3 | 6.7 min |
| 4 | 7.6 min |

The monomeric aluminum chloride hexahydrate shows only one peak at 8.1 min in the chromatogram. Polymeric aluminum species thus move within the exclusion volume of the separating column and are therefore eluted and detected first. To characterize the novel active ingredients, the areas of peaks 1-4 were then compared. The results are shown in Table 5:

TABLE 5

| | | | Active | Active | Active | |
|---|---|---|---|---|---|---|
| | Basic | Aluminum | ingredient 1 | ingredient 2 | ingredient 3 | |
| | aluminum | chloride | according to | according to | according to | |
| | chloride | hexahydrate | the invention | the invention | the invention | |
| Peak 1 | 36.9% | 0 | 4.7% | 2.2% | 2.2% | Al |
| 0-5.7 min | | | | | | polymer |
| | | | | | | high MW |
| Peak 2 | 40.7% | 0 | 30.6% | 32.7% | 31.5% | Al |
| 5.7-6.5 min | | | | | | polymer |
| | | | | | | moderate |
| | | | | | | MW |
| Peak 3 | 10.5% | 0 | 30.3 | 32.3% | 32.5% | Al |
| 6.5-7.2 min | | | | | | oligomer |
| Peak 4 | 8.1% | 35% | 31.7% | 30.8% | 31.7% | Al |
| 7.2-8.1 min | | | | | | monomer |

The table clearly shows that with the novel active ingredient the fractions of Al polymer with high and moderate molecular weight are significantly reduced and at the same time the fractions of Al monomer and Al oligomer greatly increase. In parallel to this, the peak maxima show a shift to lower molecular weights.

TABLE 6

| | Basic | Aluminum | | Active | Active | Active |
|---|---|---|---|---|---|---|
| Peak | aluminum | chloride | | ingredient | ingredient | ingredient |
| maximum | chloride | hexahydrate | | 1 | 2 | 3 |
| Maximum | 5.27 min | 0 | | 6.53 min | 6.35 min | 6.35 min |
| 1 | | | | | | |
| Maximum | 6.11 min | 0 | | 6.95 min | 6.88 min | 6.88 min |
| 2 | | | | | | |
| Maximum | 6.68 min | 0 | | 7.20 min | 7.05 min | 7.05 min |
| 3 | | | | | | |
| Maximum | 7.57 min | 8.17 | min | 7.73 min | 7.73 min | 7.73 min |
| 4 | | | | | | |

### Example 6

The novel aluminum complex described in Example 1 was further processed together with zirconium salt to give an antiperspirant mixture.

TABLE 7
Zirconium-Al complex

| Fraction | Amount |
|---|---|
| Novel Al complex from Example 1 | 14.6 g |
| Basic zirconium carbonate | 4.3 g |
| Hydrochloric acid | 1.6 g |
| Glycine | 1.1 g |

Firstly, the basic zirconium carbonate was dissolved in hydrochloric acid, glycine was added and the resulting mixture was added, with stirring, to the novel antiperspirant Al complex from Example 1.

The resulting active ingredient combination was analyzed with regard to its molecular weight distribution using GPC. The separating column used was an RP1 system. The eluent used was HNO₃/water, with the change in the refractive index being detected. The reference used was standard commercial zirconium aluminium glycine tetrachlorohydrate.

TABLE 8
Active ingredient 1 combined with zirconium

| | | Zr—Al complex | |
|---|---|---|---|
| | ZAG | from Ex. 6 | |
| Peak 1 | 19.5% | 12.0% | Al polymer |
| 0-5.7 min | | | high MW |
| Peak 2 | 40.5% | 52.0% | Al polymer |
| 5.7-6.5 min | | | moderate MW |
| Peak 3 | 2.9% | 12.4 | Al oligomer |
| 6.5-7.2 min | | | |
| Peak 4 | 10.5% | 19.3% | Al monomer |
| 7.2-8.1 min | | | |

Table 8 shows that the novel active ingredient complex with the complex ligands according to the invention shows reduced fractions of high polymer, while Al oligomer and monomer species increase considerably.

### Example 7

The novel aluminum complex described in Example 2 was further processed together with zirconium salt to give an antiperspirant mixture.

TABLE 9
Zirconium-Al complex

| Fraction | Amount |
|---|---|
| Novel Al complex from Example 2 | 13.6 g |
| Basic zirconium carbonate | 4.3 g |
| Hydrochloric acid | 1.8 g |
| Glycine | 1.1 g |

Firstly, the basic zirconium carbonate was dissolved in hydrochloric acid, glycine was added and the resulting mixture was added, with stirring, to the novel antiperspirant Al complex from Example 1.

The resulting active ingredient combination was analyzed with regard to its molecular weight distribution using GPC. The separating column used was an RP1 system. The eluent used was HNO₃/water, with the change in the refractive index being detected. The reference used was standard commercial zirconium aluminium glycine tetrachlorohydrate.

TABLE 10
Active ingredient 2 combined with zirconium

| | | Zr—Al complex | |
|---|---|---|---|
| | ZAG | from Ex. 7 | |
| Peak 1 | 19.5% | 4.4% | Al polymer |
| 0-5.7 min | | | high MW |
| Peak 2 | 40.5% | 43.6% | Al polymer |
| 5.7-6.5 min | | | moderate MW |
| Peak 3 | 2.9% | 21.7 | Al oligomer |
| 6.5-7.2 min | | | |
| Peak 4 | 10.5% | 24.6% | Al monomer |
| 7.2-8.1 min | | | |

Table 10 shows that the novel active ingredient complex with the complex ligands according to the invention shows reduced fractions of high polymer, while Al oligomer and monomer species increase considerably.

1. A process for producing a basic aluminum halide complex comprising the step of dissolving an aluminum metal with hydrohalic acid and an organic acid.

2. A process according to claim 1, wherein the hydrohalic acid is hydrochloric acid.

3. A process according to claim 1, wherein the organic acid is a hydroxycarboxylic acid.

4. A process according to claim 1, wherein the organic acid is glycolic acid or lactic acid.

5. A process according to claim 1, wherein the process is carried out at a temperature of from 20 to 100° C.

6. A process for producing an basic aluminum halide complex comprising the steps of dissolving an aluminum metal with a mixture of a hydrohalic acid and an organic acid and subsequently adding a hydrohalic acid, an organic acid or both.

7. A mixture of at least one basic aluminum halide complex produced by the process according to claim 1 and at least one antiperspirant active ingredient.

8. A process for producing a basic aluminum halide complex comprising the steps of dissolving an aluminum metal with hydrohalic acid and subsequently adding an organic acid.

9. A process according to claim 8, wherein the hydrohalic acid is hydrochloric acid.

10. A process according to claim 8, wherein the organic acid is a hydroxycarboxylic acid.

11. A process according to claim 8, wherein the organic acid is glycolic acid or lactic acid.

12. A process according to claim 8, wherein the process is carried out at a temperature of from 20 to 100° C.

13. A basic aluminum halide complex produced by the process according to claim 1.

14. A basic aluminum halide complex produced by the process according to claim 6.

15. A basic aluminum halide complex produced by the process according to claim 8.

16. An antiperspirant comprising a basic aluminum halide complex produced by the process according to claim 1.

17. An antiperspirant comprising a basic aluminum halide complex produced by the process according to claim 6.

18. An antiperspirant comprising a basic aluminum halide complex produced by the process according to claim 8.

19. A mixture of at least one basic aluminum halide complex produced by the process according to claim 6 and at least one antiperspirant active ingredient.

20. A mixture of at least one basic aluminum halide complex produced by the process according to claim 8 and at least one antiperspirant active ingredient.
